(19) United States
(12) Patent Application Publication
(10) Pub. No.: US 2015/0368620 A1
(43) Pub. Date: Dec. 24, 2015

(54) METHOD OF DETACHING ADHERENT CELLS FOR FLOW CYTOMETRY

(71) Applicant: KING ABDULLAH UNIVERSITY OF SCIENCE AND TECHNOLOGY, Thuwal (SA)

(72) Inventors: Mandeep Kaur, Thuwal (SA); Luke E. Esau, Cape Town (ZA)

(21) Appl. No.: 14/764,357

(22) PCT Filed: Jan. 29, 2014
(86) PCT No.: PCT/IB2014/001133

§ 371 Date: Jul. 29, 2015

Related U.S. Application Data
(60) Provisional application No. 61/758,527, filed on Jan. 30, 2013.

(51) Int. Cl. C12N 5/09; G01N 15/14; C12N 5/071

(52) U.S. Cl. CPC C12N 5/0693; C12N 5/0625; G01N 15/14; C12N 2509/00; G01N 2015/0065

(57) ABSTRACT

In one aspect, a method for detaching adherent cells can include adding a cell lifting solution to the media including a sample of adherent cells and incubating the sample of adherent cells with the cell lifting solution. No scraping or pipetting is needed to facilitate cell detachment. The method do not require inactivation of cell lifting solution and no washing of detaching cells is required to remove cell lifting solution. Detached cells can be stained with dye in the presence of cell lifting solution and are further analyzed using flow cytometer. The method has been tested using 6 different cell lines, 4 different assays, two different plate formats (96 and 384 well plates) and two different flow cytometry instruments. The method is simple to perform, less time consuming, with no cell loss and makes high throughput flow cytometry on adherent cells a reality.

### PRIORITY CLAIM

This application claims priority to U.S. Provisional Application No. 61/758,527, filed Jan. 30, 2013, which is incorporated by reference in its entirety.

### TECHNICAL FIELD

This description relates to a method of detaching adherent cells for flow cytometry.

### BACKGROUND

Flow cytometry can provide a powerful and accurate measure of cellular information down to the single cell and can be used to measure apoptosis in cells. Generally, flow cytometry requires cells to be in suspension as they pass through tubing leading cells through the various lasers and detectors. This in itself can be one of the limitations of flow cytometry, as test experiments have to be in suspension. For this reason suspension cell lines can be preferred, as they provide a platform immediately compatible with high throughput flow cytometry. Adherent cells can be cultured in suitable numbers and trypsinized to obtain a cell suspension suitable for use in flow cytometry. Working with adherent cells can, thus, limit the potential for high throughput flow cytometry assays because downstream processing including washing and trypsinisation can result in cell loss even before reaching the flow cytometer.

### SUMMARY

In one aspect, a method for detaching adherent cells can include adding a cell lifting solution to a media including a sample of adherent cells and incubating the sample of adherent cells with the cell lifting solution.

In certain embodiments, the sample of adherent cells can include HeLa cells, DU145 cells, HT1080 cells, BT20 cells, MCF-7 cells or BJ fibroblasts. The sample of adherent cells can be incubated with the cell lifting solution for five minutes or more. The sample of adherent cells can be incubated with the cell lifting solution for fifteen minutes or more. The sample of adherent cells can be incubated with the cell lifting solution for thirty minutes or more.

In certain embodiments, the cell lifting solution can include a metal ion chelator. The metal ion chelator can be EDTA. The cell lifting solution can be free of enzyme.

In another aspect, a method for detaching adherent cells for flow cytometry can include adding a cell lifting solution to a media including a sample of adherent cells, incubating the sample of adherent cells with the cell lifting solution, and analyzing the sample of adherent cells by flow cytometry.

In certain embodiments, the method can further comprise adding an indicator of apoptosis to the sample of adherent cells.

In certain embodiments, the indicator of apoptosis can be a dye, a fluorophore or an antibody.

In certain embodiments, the method can further comprise adding a stimulus to the media. The stimulus can be a drug or extract.

In certain embodiments, the adherent cells are not washed with a washing solution between adding the cell lifting solution to the media and analyzing the adherent cells by flow cytometry.

In certain embodiments, the time period between adding the cell lifting solution to the media and analyzing the adherent cells by flow cytometry can be four hours or less. The time period between adding the cell lifting solution to the media and analyzing the adherent cells by flow cytometry can be three hours or less. The time period between adding the cell lifting solution to the media and analyzing the adherent cells by flow cytometry can be two hours or less. The time period between adding the cell lifting solution to the media and analyzing the adherent cells by flow cytometry can be one hour or less. The time period between adding the cell lifting solution to the media and analyzing the adherent cells by flow cytometry can be 30 minutes or less, for example, 15-30 minutes.

In certain embodiments, the stimulus can be added to the media prior to adding the cell lifting solution to the media.

In certain embodiments, the cell lifting solution can include a metal ion chelator. The metal ion chelator can be EDTA. The pH of the cell lifting solution can be betweek 6.1 and 6.4.

In certain embodiments, the cells are not washed using centrifugation after lifting. The cells can be stained with a dye/stain and subjected to flow cytometry.

In another aspect, a method of collecting adherent cells can include plating a first sample of adherent cells in media on a cell culture plate; plating a second sample of adherent cells in media on a cell culture plate, wherein the second sample of adherent cells includes a second number of adherent cells; adding a control buffer to the media of the first sample of adherent cells; adding a stimulus to the media of the second sample of adherent cells; adding cell lifting solution to the media of the first sample of adherent cells and to the media of the second sample of adherent cells, wherein the first sample includes a first number of adherent cells at the time the cell lifting solution is added to the media and the second sample includes a second number of adherent cells at the time the cell lifting solution is added to the media; incubating the first and second samples of adherent cells, thereby detaching the first and second samples of adherent cells from the cell plate; collecting the first sample of adherent cells, wherein the number of adherent cells collected from the first sample is equal to 75% or more of the number of adherent cells in the first sample when the cell lifting solution was added; and collecting the second sample of adherent cells, wherein the number of adherent cells collected from the second sample is equal to 75% or more of the number of adherent cells in the second sample when the cell lifting solution was added.

### BRIEF DESCRIPTION OF THE DRAWINGS

FIG. 1 is a flow diagram outlining a method for detaching adherent cells for flow cytometry.

FIG. 2A is a graph showing the separation of untreated and treated BJ cells using flow cytometer following the performance of a cell detachment method.

FIG. 2B is a 1D histogram of untreated and treated BJ cells following the performance of a cell detachment method.

FIG. 2C is a bar graph showing the percentage of non-apoptotic and apoptotic cells within the untreated or treated population of BJ cells following the performance of a cell detachment method.

FIG. 3A is a graph showing the separation of untreated and treated MCF-7 cells following the performance of a cell detachment method.

FIG. 3B is a 1D histogram of untreated and treated MCF-7 cells following the performance of a cell detachment method.

FIG. 3C is a bar graph showing the percentage of non-apoptotic and apoptotic cells within the untreated or treated population of MCF-7 cells following the performance of a cell detachment method.

FIG. 4A is a graph showing the separation of untreated and treated HeLa cells following the performance of a cell detachment method.

FIG. 4B is a 1D histogram of untreated and treated HeLa cells following the performance of a cell detachment method.

FIG. 4C is a bar graph showing the percentage of non-apoptotic and apoptotic cells within the untreated or treated population of HeLa cells following the performance of a cell detachment method.

FIG. 5A shows separation of untreated (blue) and treated (red) cells based on FL2-H and SSC-H profile; FIG. 5B shows 1D Histogram of untreated (blue) and treated (red) cells measuring fluorescence in the FL2-H channel and applying gates to delineate Non-apoptotic (blue) and Apoptotic (red) cells; FIG. 5C is bar graph showing the percentage of non-apoptotic and apoptotic cells within the untreated or treated (positive control) population of cells.

FIG. 6 shows 1D Histogram of untreated (blue) and treated (red) cells measuring fluorescence in the PE-A channel and applying gates to delineate Non-apoptotic (blue) and Apoptotic (red) cells as measured using BD LSRFortessa HTS flow cytometer.

FIG. 7A shows separation of unstained (blue), untreated (red) and treated (green) cells based on FL2-H and FL-1-H profile (A); FIG. 7B shows 1D Histogram of unstained (blue), untreated (red) and treated (green) cells measuring fluorescence in the FL2-H channel (B) as measured using BD LSRFortessa HTS flow cytometer.

FIG. 8 shows untreated (red) and treated cells for MMP disruption in DU145 cells as measured using BD LSRFortessa HTS flow cytometer.

FIGS. 9A-B shows analysis of HeLa and MCF-7 cells by flow cytometry on an Accuri C6 to determine the level of ROS in cells.

FIGS. 10A-B shows untreated (blue peak) and treated cells (red peak) for ROS as measured using BD LSRFortessa HTS flow cytometer.

FIG. 11A shows the percentages of cells after various wash steps for DU145 cells, and FIG. 11B) shows results for HeLa cells. This data compares the retained cell number in conventionally used method as compared to our method.

### DETAILED DESCRIPTION

Apoptosis is a form of programmed cell death that plays an important role in normal homeostasis of dividing cells and their development. Apoptosis also can play a role in diseases, such as cancer. Cancer cells have the ability to avoid apoptosis which thus presents an interesting event to quantify when determining the ability of drugs to induce apoptosis in cancer cells. Features of apoptosis can include phosphatidyl serine exposure, activation of endonucleases leading to DNA fragmentation, nuclear condensation, blebbing and mitochondrial changes resulting in loss of mitochondrial membrane potential and release of cytochrome c. (Denecker G, et al.: Phosphatidyl serine exposure during apoptosis precedes release of cytochrome c and decrease in mitochondrial transmembrane potential. FEBS Lett, 2000, 465:47-52; Yivgi-Ohana N, et al.: Utilizing mitochondrial events as biomarkers for imaging apoptosis. Cell Death Dis, 2011, 2:e166; Bertho A L, et al.: Flow cytometry in the study of cell death. Mem Inst Oswaldo Cruz, 2000, 95:429-433; Wlodkowic D, et al.: Cytometry of apoptosis. Historical perspective and new advances. Exp Oncol, 2012, 34:255-262, each of which is incorporated by reference in its entirety.) Some of these indicative features of apoptosis can act as markers to determine the state of apoptosis in a population of cells.

Flow cytometry is a process by which cells in suspension can be flowed through a fluid handling device and characterized and separated by microfluidic processes according to their light scattering properties. In this technique, cells can be individually passed through a laser in a stream of isotonic saline, for example. Detection of the light scattering properties can allow a computer to characterize each cell according to its size and structure. In a heterogeneous population of cells, the proportion of each type of cell can be determined, and it can even be possible through rapid gating to separate distinct populations into individual receptacles in the course of an analysis. In addition to straightforward light scattering properties, cells can be labeled with fluorescent antibodies, allowing a flow cytometer to detect protein expression levels within cells. Multiple antibodies can be used. In extreme cases, flow cytometry can have the capability to measure 18 fluorescent colors in a single analysis. The data collected can be displayed on an X/Y axis histogram. For example, size can be charted versus structural complexity; in the case of fluorescence data collection, green-labeled protein expression can be charted against another protein that is labeled with a red label/marker, allowing discernment of relationships between protein pathways according to the cell's environmental conditions.

Because of the nature of the technique, in which cells are free-floating in solution as they pass through the laser, flow cytometry was originally applied to cells that only grew in suspension such as blood-derived cell types. However, this technique has now been applied to adherent cells as well. The technique obviously requires lifting adherent cells from their substrate so they can be carried in fluid through the laser. Considerable debate revolves around whether the act of removing cells from their natural substrate can introduce artifacts into cell data collection.

Flow cytometry has been used mostly as a basic research tool and has been regarded as a labor intensive procedure, for which the data sets gathered can be cumbersome to process. In many labs, it has been traditional to manually feed a cell suspension in a test tube to a “sipper” that draws each sample into the flow cytometry machine. A lack of sample standardization, a taxing sample preparation method, and the generation of large amounts of data meant flow cytometry was more suited to preparing basic research papers than industrial biotechnology applications. Recently, however, improvements in data management and adaptation of flow cytometry apparatuses to robotically collected and analyzed cells from 96- and 384-well microplates has allowed flow cytometry to be used for large pharma-scale research. Flow cytometry can have enormous potential in this regard; the capability to simultaneously measure two to ten different protein expression patterns, as well as basic cell phenotype, makes flow cytometry ideal for characterizing cellular response to changing environmental conditions such as exposure to therapeutic candidates in a drug screening campaign.

Many drug discovery campaigns focus on apoptosis, as activation of this cell death pathway is seen as a key outcome for anti-cancer therapeutics. A hallmark feature of adherent cells can be their natural tendency to detach from their substrate as apoptosis progresses. Flow cytometry can rely on standard immunofluorescence protocols, in which the growth medium can be removed from a layer of cells, a fluorescently tagged antibody can be added to the cells, and then several washes can be applied to remove unbound antibody. In a protocol involving apoptosis detection, a prominent concern can be that medium aspiration and subsequent washes can remove detached and loosely adhered apoptotic cells, a potentially essential cell population to characterize in any screening campaign seeking to assess cytotoxicity.

In other words, flow cytometry is a technique that can provide a powerful and accurate measure of cellular information down to the single cell and is widely used to measure apoptosis in the cells. Flow cytometry can use a machine built on the principal of detecting signals/information by employing lasers to excite fluorophores and detectors to acquire emissions given off by flours. Flow cytometry can require cells to be in suspension as they pass through tubing, leading the cells through various lasers and detectors. For this reason, suspension cell lines can be preferred as they provide a platform immediately compatible with high throughput flow cytometry. (Black C B, et al.: Cell-based screening using high-throughput flow cytometry. Assay Drug Dev Technol, 2011, 9:13-20, which is incorporated by reference in its entirety). Adherent cells can be cultured in required numbers and can be trypsinized to obtain a cell suspension suitable for use in flow cytometry. Routinely used protocols include various wash and centrifugation steps that make protocols cumbersome and time consuming. Working with adherent cells can, thus, limit the potential for high throughput flow cytometry assays, because downstream processing, including washing and trypsinisation, can result in cell loss even before reaching the flow cytometer.

Accordingly, it would be advantageous to develop a protocol that allows the lifting of adherent cells without removing media and eliminate wash steps. Protocols described herein overcome these disadvantages. The methods are advantageous because adherent cells can now be used for medium to high throughput flow cytometry, for example in 96 well plate scale.

Adherent cells refers to cells, cell lines, and cell systems, whether prokaryotic or eukaryotic, that remain associated with, immobilized on, or otherwise in contact with the surface of a substrate, and remain so through washing or medium exchange procedures. Examples of cells that can be grown as adherent cells or immobilized on a surface are liver or liver-derived cells including primary hepatocytes and liver epithelial cells, epithelial cells in general, endothelial cells in general, neuronal cells, mesenchymal cells, pancreatic cells, skeletal muscle cells, cardiomyocytes, carcinoma-derived cells, bone marrow cells, islets of Langerhans, adrenal medulla cells, osteoblasts, osteoclasts, T-lymphocytes, neurons, glial cells, ganglion cells, retinal cells, and myoblast cells. Stem cells can also be used; examples are mesenchymal stem cells, neuronal stem cells, induced pluripotent stem cells, hematopoietic stem cells, mouse embryonic stem cells, and human embryonic stem cells. Specific examples of adherent cells include HeLa (cervical carcinoma), HT1080 (Colon carcinoma), DU145 (Prostate cancer), MCF-7 and BT20 (Breast cancer) and BJ (normal fibroblasts). The sample of adherent cells can be incubated with the cell lifting solution for five minutes or more, fifteen minutes or more, thirty minutes or more, or one hour or more.

A growth medium or culture medium is a liquid or gel designed to support the growth of microorganisms or cells. There are two major types of growth media: those used for cell culture, which use specific cell types derived from plants or animals, and microbiological culture, which are used for growing microorganisms, such as bacteria or yeast. The most common growth media for microorganisms are nutrient broths and agar plates; specialized media are sometimes required for microorganism and cell culture growth. Some organisms, termed fastidious organisms, require specialized environments due to complex nutritional requirements. Viruses, for example, are obligate intracellular parasites and require a growth medium containing living cells. There are different types of media for growing different types of cells. Media that can be used to grow cells include Dulbecco's Modified Eagle Medium, Minimum Essential Media, RPMI Medium 1640, Iscove's Modified Dulbecco's Medium, F10 Nutrient Mixture, Ham's F12 Nutrient Mixture, and so on. Additionally, the media can include salts, amino acids, Fetal bovine serum, or Fetal Calf Serum.

A cell lifting solution can be used to dissociate adherent cells. The cell lifting solution can be free of enzyme and can contain a chelator or a mixture of chelators. A chelator is a chemical compound in the form of a heterocyclic ring, containing a metal ion attached by coordinate bonds to at least two nonmetal ions. Examples of chelator include ethylenediaminetetraacetic acid (EDTA), ethylene glycol tetraacetic acid (EGTA), porphine, heme, ethylenediamine, or dimercaprol.

Flow cytometers are able to analyze several thousand cells every second, in “real time,” and can actively separate and isolate cells having specified properties. A flow cytometer is similar to a microscope, except that, instead of producing an image of the cell, flow cytometry offers “high-throughput” (for a large number of cells) automated quantification of set parameters. Adherent cells can be prepared for high throughput flow cytometry. Samples can be measured at a rate of up to 40 wells per minute. In addition, cells, such as BJ cells, can be seeded at a density of 5×10³ cells per well with 96 well plates.

Recovery is the proportion of sorted cells of interest compared to the total number of cells of interest. In certain embodiments, percent of cells, such as adherent cells, recovered at the end can be 75% or more, 80% or more, 85% or more, 90% or more, or 95% or more.

In certain embodiments, the time period between adding the cell lifting solution to the media and analyzing the adherent cells by flow cytometry can be four hours or less, three hours or less, two hours or less, or one hour or less.

Conventional methods of flow cytometry often require removing media from cells, and lifting of the cells using solutions that often needs pipetting and may also require cell scraping. In the method here, the cells can be lifted without pipetting or scraping, the media do not have to be removed from wells, and the cell lifting solution can be added directly to the wells. In addition, the method here does not require washing or centrifugation step, and is less cumbersome and time consuming than the conventional protocol that requires washing or centrifugation step.

In addition, because the media does not have to be removed, the method here minimizes cells' time out of media, which lowers the baseline of apoptotic cells. The method here also minimizes cell trauma as neither scraping is required to lift cells nor harsh lifting solutions like trypsin are used. In addition, the method here can achieve effective separation. The percent of cells separated to single cells can be 75% or more, 80% or more, 85% or more, 90% or more, or 95% or more

### Examples

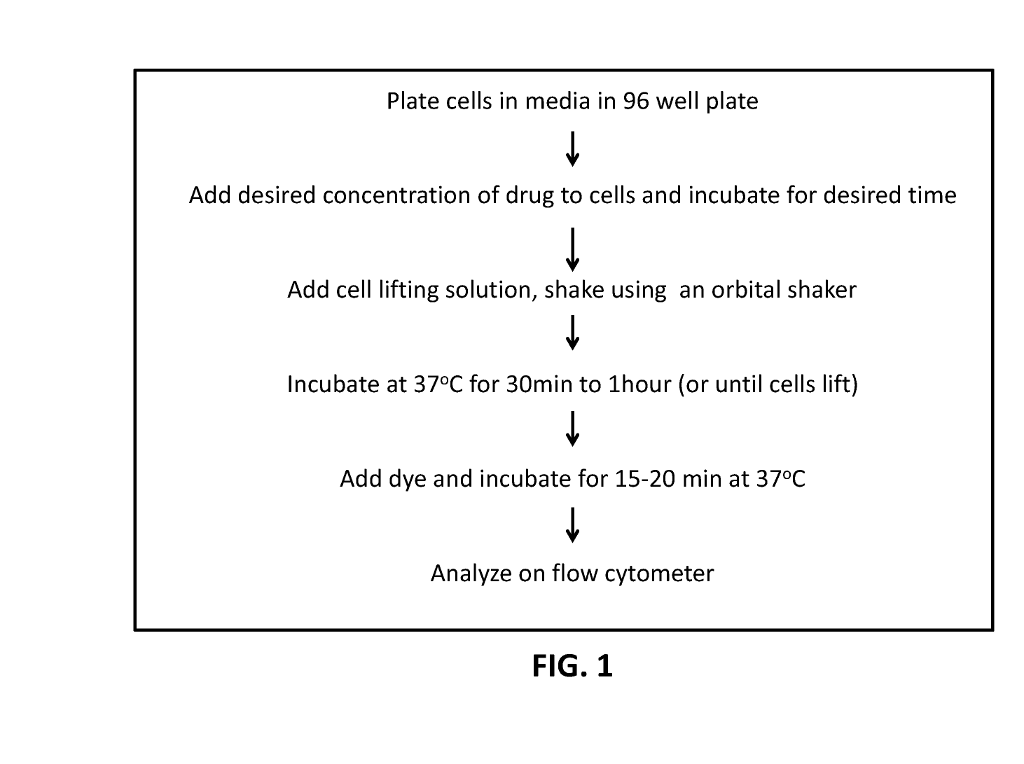

FIG. 1 illustrates the steps performed in one embodiment. As shown, cells are first plated in media. A control sample or drug sample can be added to the cells. The concentration of the drug sample and the time the cells are exposed to the drug sample can vary. The cell lifting solution can be added to the cells and incubated until the adherent cells lift from the culture container. A dye can be added to the cells prior to the cells being analyzed by flow cytometry. The protocol can be used to study the apoptosis induced after treatment with drug sample, for example, an apoptosis inducer.

In one method, five thousand cells were plated per well in 45 μl of media in 96-well plate. The cells were shaken at 750 rpm with an Orbital shaker for 20 seconds and allowed to settle for 16-24 hrs in 5% CO₂ incubator at 37° C. A desired concentration of drug was added to cells in a volume of 50 to make the final volume per well up to 50 μl. The cells and drug were mixed at 750 rpm with an Orbital shaker for 20 seconds and incubated again for desired period of time. Twelve microliters of 15 mM EDTA pH 6.14 (upto 20 mM can be added depending on the cell line) were added per well, mixed at 750 rpm with an Orbital shaker for 20 seconds and incubated at 37° C. for 45 min to 1 hour (until cells lift). Fifteen microliters of media containing 1 μl APOPercentage dye was added to the cells and incubated for 15-20 min at 37° C. The cells were analyzed on flow cytometer setting a threshold for FSC-H of 80,000, cytometer speed at medium (2 μl/sec), peristaltic pump speed at 23 rpm, shaking on 1800 rpm, gating on 2D plot of FSC vs SSC and a 1D plot measuring FL2-H/FL3-H on the X axis. The HTFC system was used.

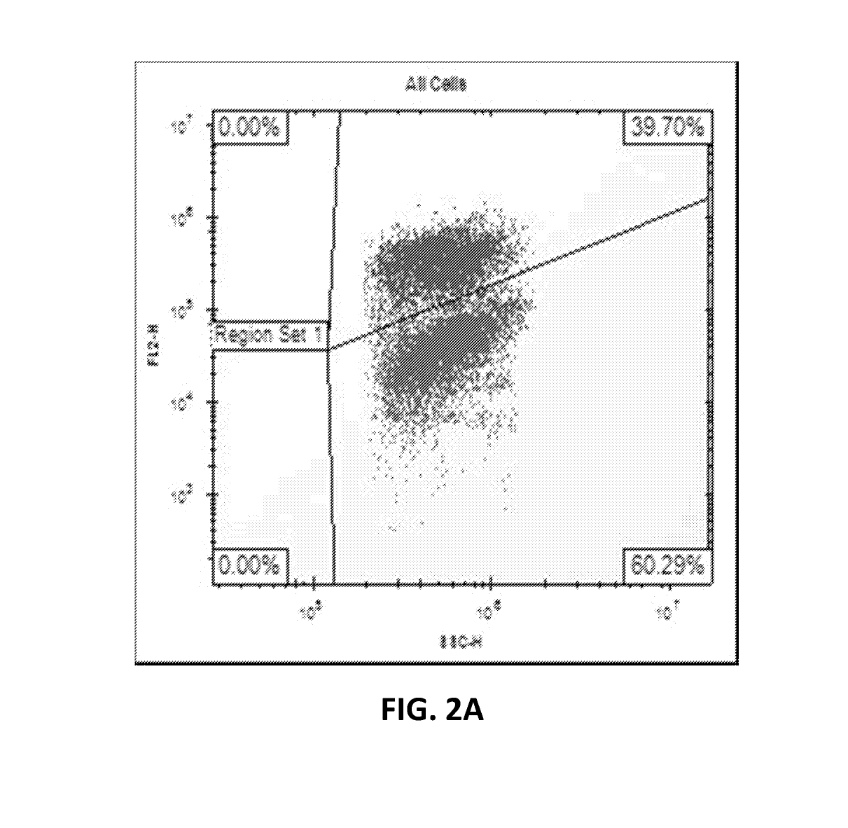

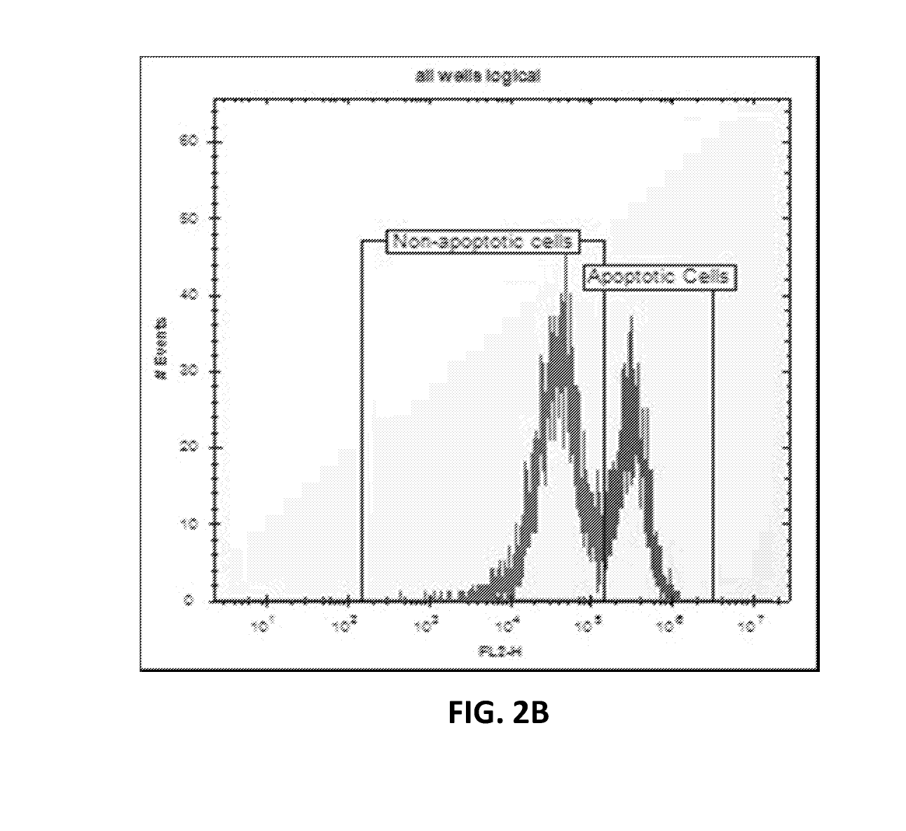

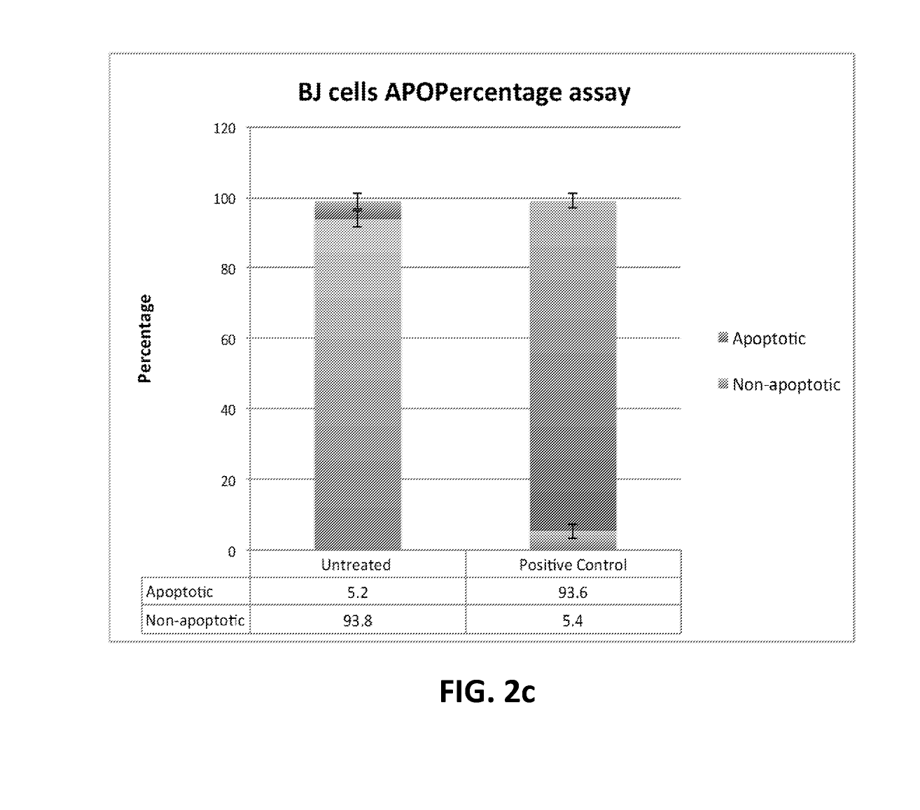

FIGS. 2, 3 and 4 shows the results obtained using three different cell lines BJ, MCF-7 and HeLa, respectively, using the protocol shown in FIG. 1. The apoptotic cells were stained with the APOPercentage dye due to flipping of cell membrane, whereas non-apoptotic cells could not take up this dye due to a normal cell membrane.

BJ fibroblast cells were analyzed by flow cytometry on an Accuri C6 flow cytometer to determine the percentage of cells undergoing apoptosis (FIG. 2). Cells were seeded at a density of 5×10³ cells per well in 96 well plate. Cells exposed to 2.5 mM H₂O₂ for 30 minutes or 62 μl EDTA pH 6.14 for 15-30 min. added to cells after removing media were used as a positive control for apoptosis. Cells were lifted and stained with 1 μl APOPercentage dye. The cells were then analyzed by flow cytometry. As shown in FIG. 2A, untreated and treated cells were separated based on FL2-H and SSC-H profile. FIG. 2B includes a 1D Histogram of untreated and treated cells showing the fluorescence measured in the FL2-H channel, where gates were applied to delineate non-apoptotic and apoptotic cells. FIG. 2C is a bar graph showing the percentage of non-apoptotic and apoptotic cells that was measured within the untreated or treated (positive control) population of cells.

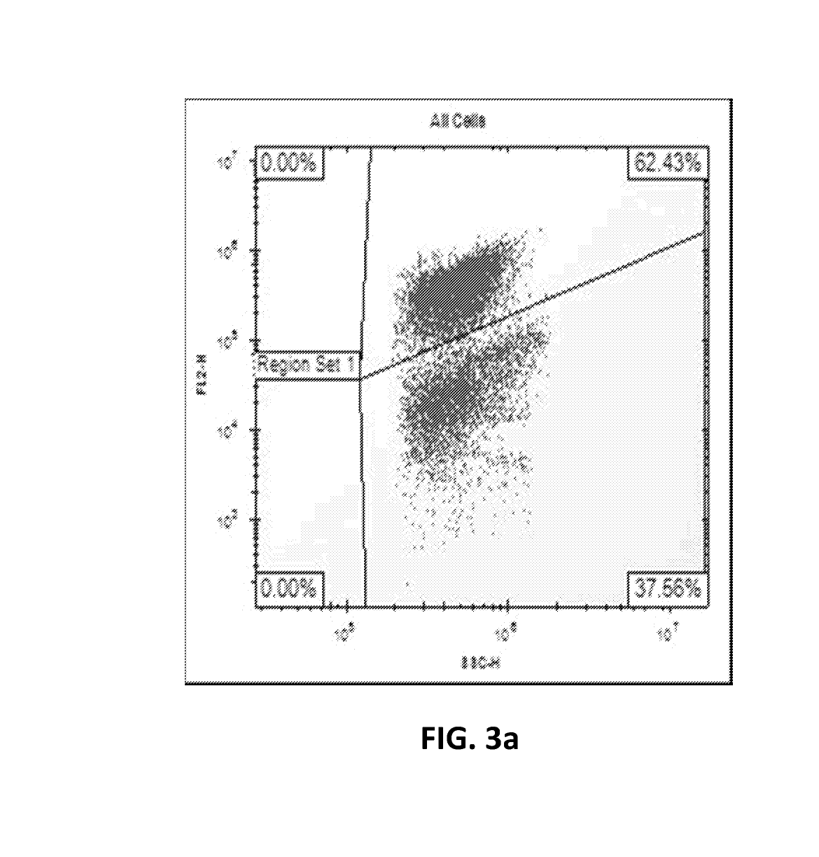

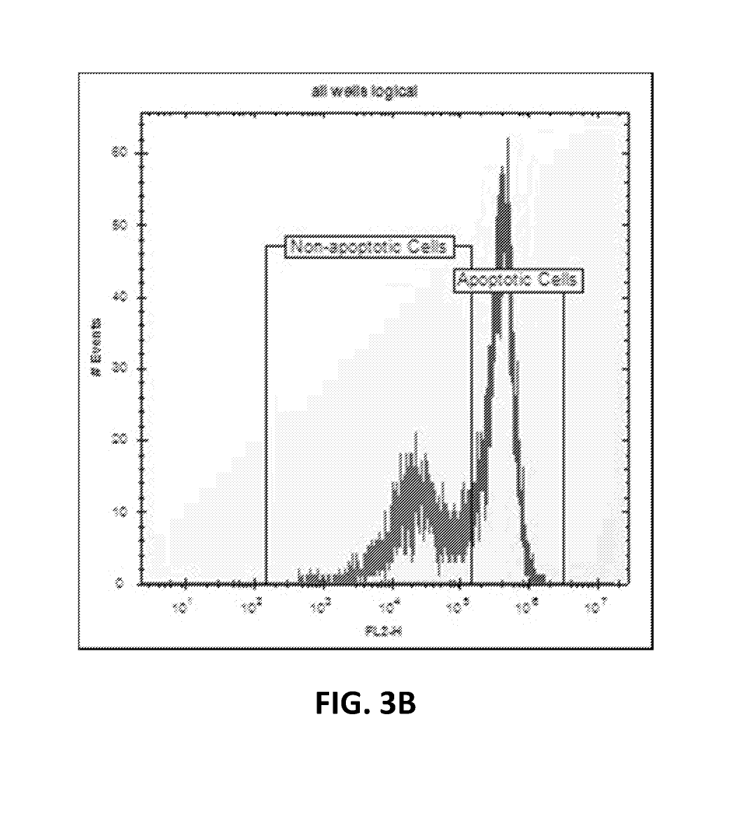

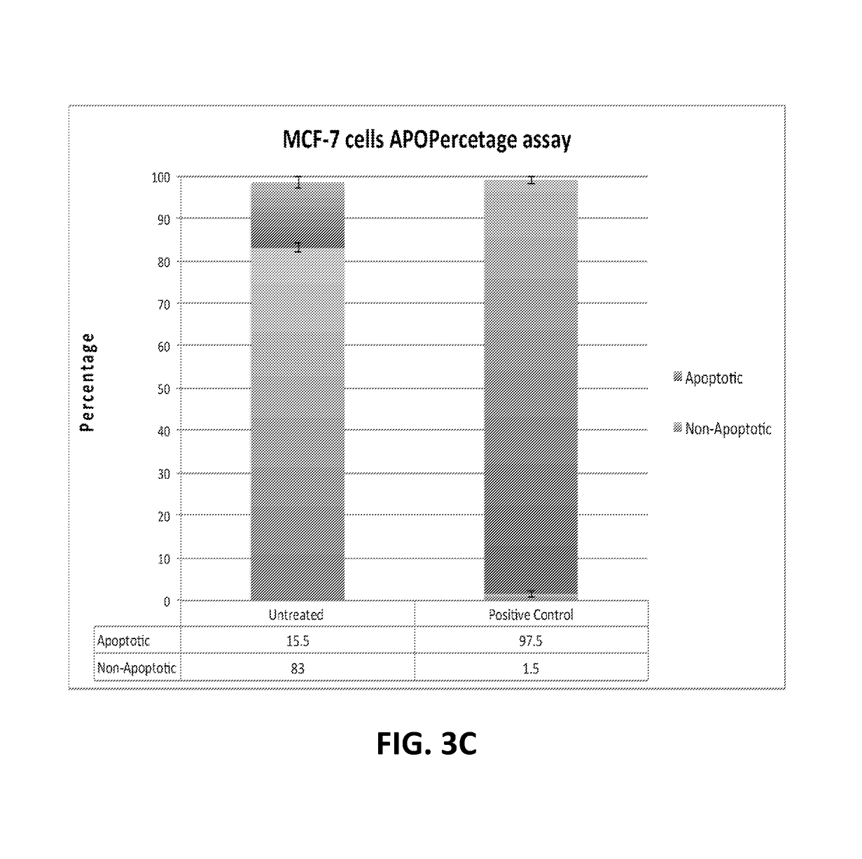

Untreated and treated MCF-7 cells were analyzed by flow cytometry on an Accuri C6 flow cytometer to determine the percentage of cells undergoing apoptosis (FIG. 3). Cells were seeded at a density of 5×10³ cells per well in 96 well plate. Cells treated with 2.5 mM H₂O₂ for 30 minutes or 62 μl EDTA pH 6.14 for 15-30 min. added to cells after removing media were used as a positive control for apoptosis. The cells were lifted and stained with 1 μl APOPercentage dye. The cells were then analyzed by flow cytometry. Separation of untreated and treated cells based on FL2-H and SSC-H profile is shown in FIG. 3A. FIG. 3B shows a 1D Histogram of untreated and treated cells showing the fluorescence measured in the FL2-H channel, where gates were applied to delineate non-apoptotic and apoptotic cells. FIG. 3C is a bar graph showing the percentage of non-apoptotic and apoptotic cells within the untreated or treated population of cells.

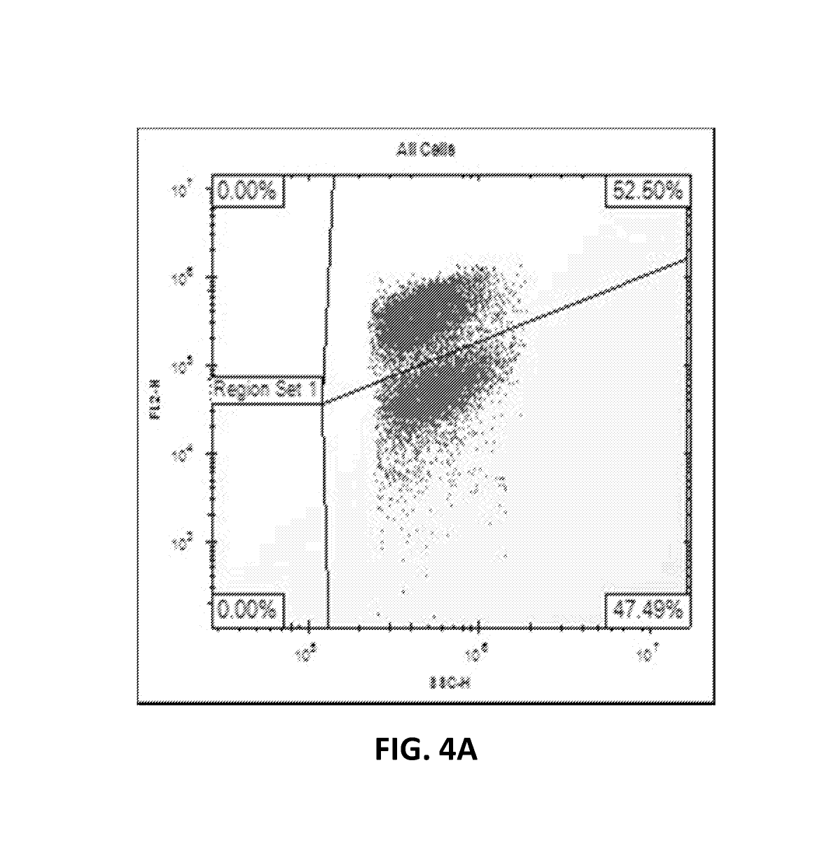

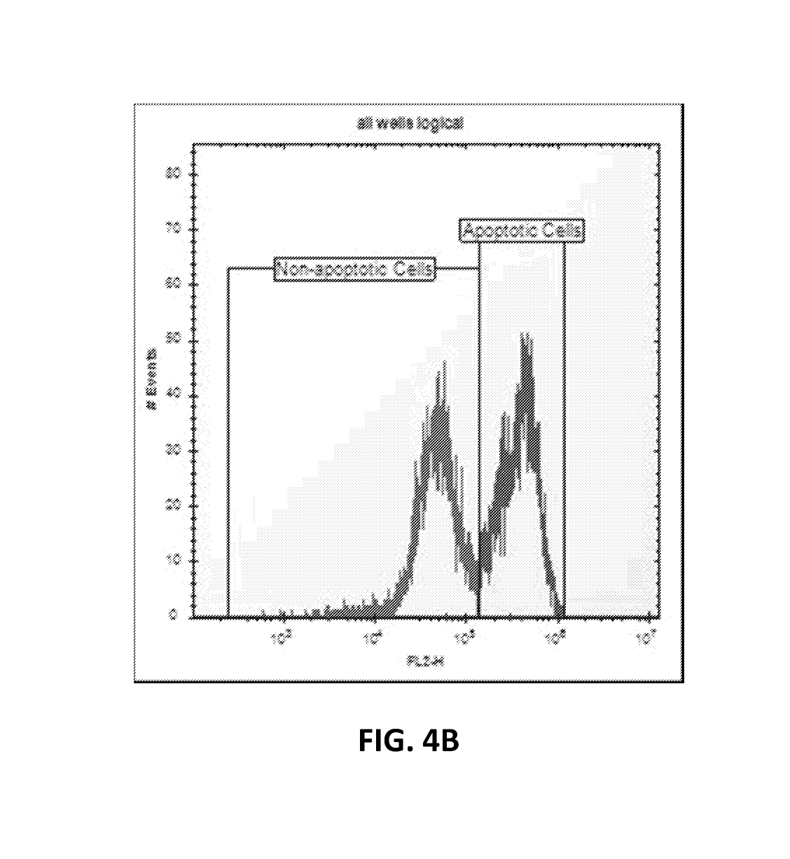

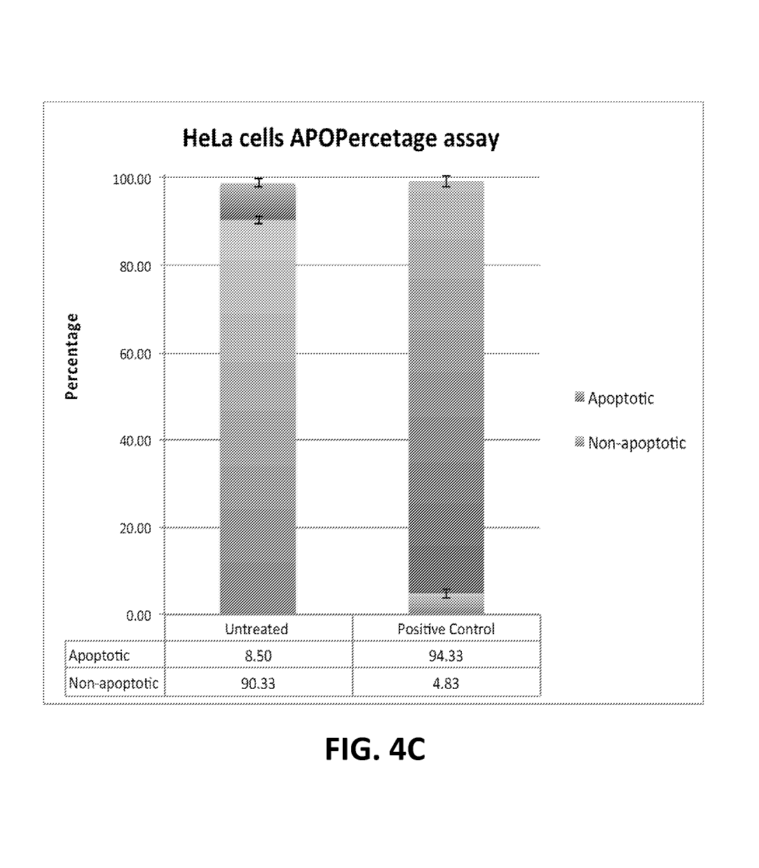

Untreated and treated HeLa cells were also analyzed by flow cytometry on an Accuri C6 flow cytometer to determine the percentage of cells undergoing apoptosis (FIG. 4). Cells were seeded at a density of 5×10³ cells per well in 96 well plate. Cells treated with 2.5 mM H₂O₂ for 30 minutes or 62 μl EDTA pH 6.14 for 15-30 min. added to cells after removing media were used as a positive control for apoptosis. Cells were lifted and stained with 10 APOPercentage dye. The cells were then analyzed by flow cytometry. Separation of untreated and treated cells based on FL2-H and SSC-H profile is shown in FIG. 4A. FIG. 4B shows a 1D Histogram of untreated and treated cells showing the fluorescence measured in the FL2-H channel, where gates were applied to delineate non-apoptotic and apoptotic cells. FIG. 4C is a bar graph showing the percentage of non-apoptotic and apoptotic cells within the untreated or treated population of cells.

The results showed that the untreated or non-apoptotic cells were unstained (i.e. did not take up dye), while cells undergoing apoptosis took up the dye. Generally, untreated cells can take up some dye as cells undergo apoptosis via a natural process, but this percentage in immortal cell line should not be as much as in cells with drug induced apoptosis. The results showed that, in the cell lines tested, the untreated cells had low numbers of positives (apoptotic cells), whereas most of the treated cells (apoptotic cells) were positively taking up the dye.

These results also showed that the protocol was stable in several cell lines tested. There was little overlap between untreated and treated cells in terms of positive staining. This confirmed that the method can have the capability to identify cells undergoing apoptosis from non-apoptotic cells. The results also confirmed that the treatment of cell lifting chemical was gentle and did not affect untreated cell membrane and also did not induce unwanted apoptosis in untreated cells.

Cell lines, such as HeLa and BJ fibroblasts, can have an elongated cell morphology. These cell lines can be more adherent and can also be more stable under assay conditions. On the other hand, the MCF-7 cells can be very sensitive and can have a tendency to respond to external factors with more sensitivity. Therefore, the percentage of cells taking up stain in untreated cells was little more using the MCF-7 cells as compared to other cell lines, but these limits were acceptable in flow cytometry.

Measuring apoptosis using APOPercentage Assay in adherent cells.

Cancer cells have the ability to avoid apoptosis, which thus presents an interesting event to quantify when determining the ability of drugs to induce apoptosis. Below the APOPercentage protocol with results is described for five cell lines (HeLa (Cervical cancer), MCF-7 (Breast Cancer), DU145 (Prostate Cancer), HT1080 (Colon Cancer), and BJ (Normal Fibroblasts). Apoptotic cells take up APOPercentage dye when membrane movement occurs and phosphatidylserine is exposed to the outer leaflet. APOPercentage dye fluoresces in the FL-2 channel with a fluorescence of 590-600 nm and can be measured by flow cytometry.

The following steps can be used to measure apoptosis using APOPercentage Assay in adherent cells. 5000 cells per well were plated in 45 μl of media in 96-well plate, shaken at 750 rpm with an Orbital shaker for 20 seconds, and allowed to settle 16-24 hours (h). Desired concentration of drug was added to cells in a volume of 50 to make the final volume per well up to 50 μl, was shaken at 750 rpm with an Orbital shaker for 20 seconds, and was then incubated for desired period of time. 12 μl of 15 mM EDTA pH 6.14 per well was added (upto 20 mM can be added depending on the cell line). Shake at 750 rpm with an Orbital shaker for 20 seconds and incubate at 37° C. for 15 minutes (min) to 1 h (until cells lift). Add 15 μl of serum free media containing 1 μl APOPercentage dye and incubate for 15-20 min at 37° C. Analyze on flow cytometer setting a threshold for FSC-H of 80 000, cytometer speed at medium (2 μl/sec), peristaltic pump speed at 23 rpm, gating on 2D plot of FSC vs SSC and a 1D plot measuring FL2-H/FL3-H on the X axis. 2.5 mM H₂O₂ is used as a positive control.

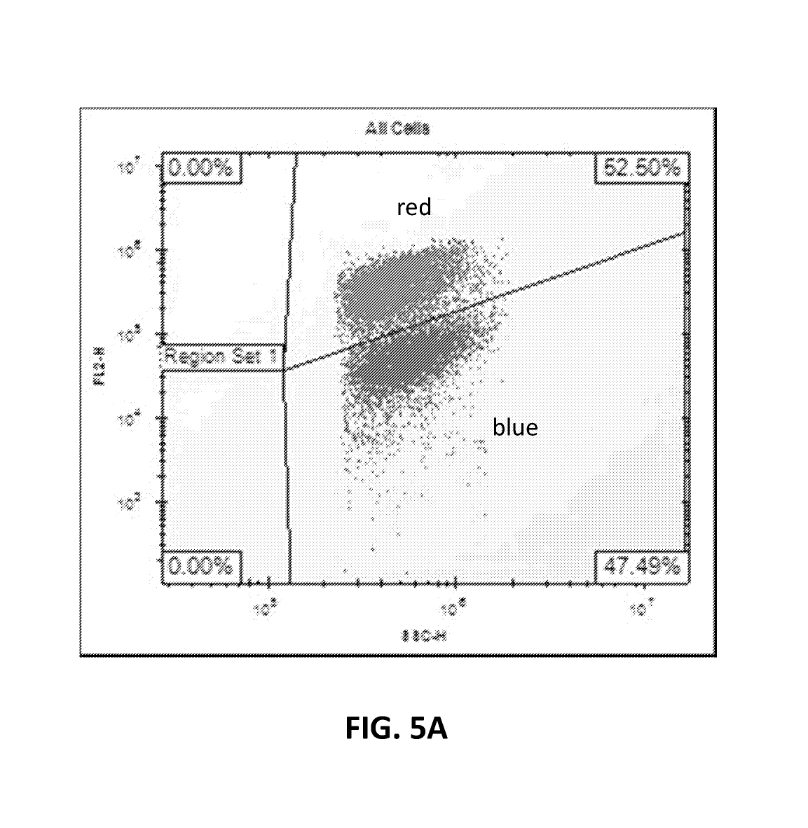

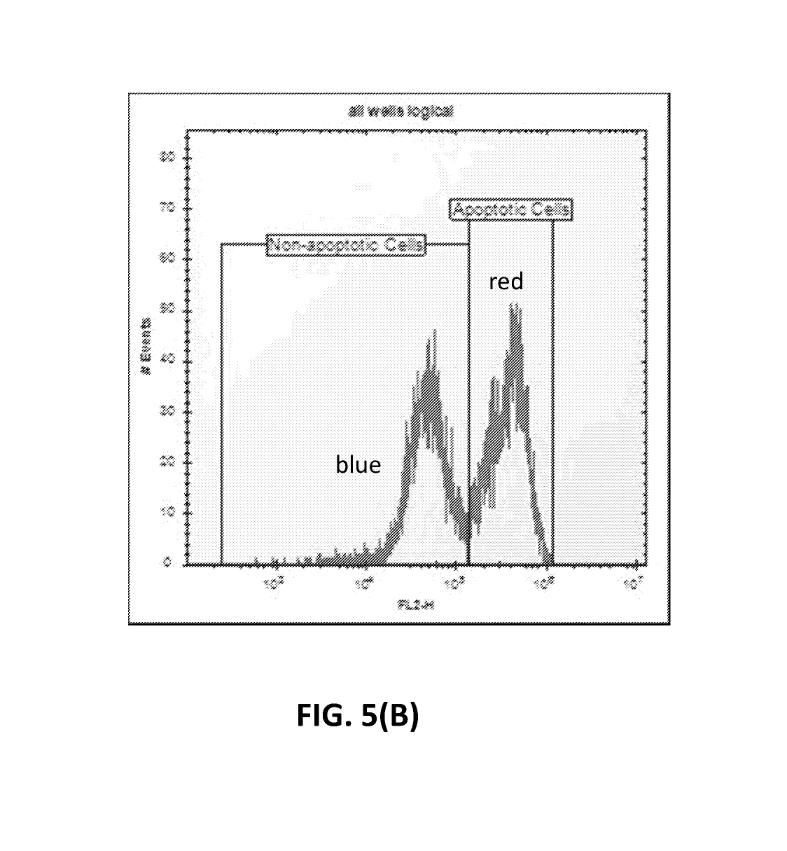

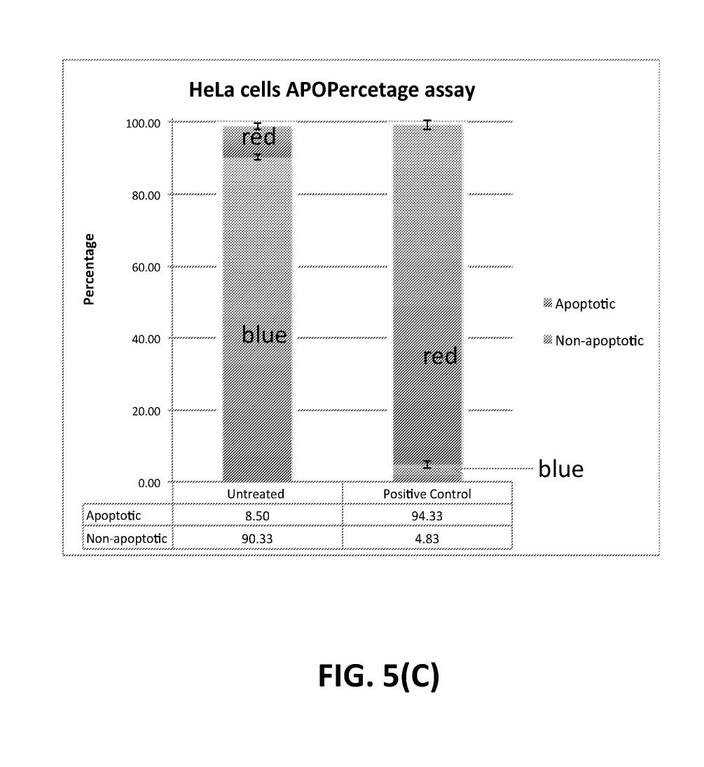

FIGS. 5A-C and Table 1 show results of APOPercentage assay in 96-well plate on Accuri C6 HTFC System (Intellicyt).

FIGS. 5A-C show that HeLa Cells untreated (blue) or treated (red) were analyzed by flow cytometry on an Accuri C6 flow cytometer to determine the percentage of cells undergoing apoptosis. Cells were seeded at a density of 5×10³ cells per well in a 96-well plate. Following day, cells were treated with 2.5 mM H₂O₂ for 30 min. or 62 μl EDTA pH 6.14 for 15-30 min. added to cells after removing media as a positive control for apoptosis for 15-30 min. Cells were lifted with EDTA and stained with 10 APOPercentage and analyzed by flow cytometry. Separation of untreated (blue) and treated (red) cells based on FL2-H and SSC-H profile (FIG. 5A). 1D Histogram of untreated (blue) and treated (red) cells measuring fluorescence in the FL2-H channel and applying gates to delineate Non-apoptotic (blue) and Apoptotic (red) cells (FIG. 5B). Bar graph showing the percentage of non-apoptotic and apoptotic cells within the untreated or treated (positive control) population of cells (FIG. 5C).

Table 1 shows different aspects of method standardized using various cell lines. ‘Stability of cells in EDTA’ refers to the time limit until the percentage of stained cells remain <10% in negative control.

TABLE 1

| | Time to lift with EDTA | Time to stain with | Stability in | Percent cells | Percent cells |
|---|---|---|---|---|---|
| | (stability of | APOPercentage | APOPercentage | stained in | stained in |
| Cell Line | cells in EDTA) | dye | dye after staining | negative control | positive control |
| DU145 | 20-30 min | 15 min | 60 min | 10 | 90 |
| | (stable up to 2 h) | | | | |
| HeLa | 30-45 min | 15 min | 60 min | 10 | 90 |
| | (stable up to 90 min) | | | | |
| MCF-7 | 15-20 min | 15 min | 45 min | 15 | 85 |
| BJ | 30-50 min | 15 min | 60 min | 10 | 90 |
| HT1080 | 20-30 min | 15 min | 60 min | 5 | 95 |
| | (stable up to 3 h) | | | | |

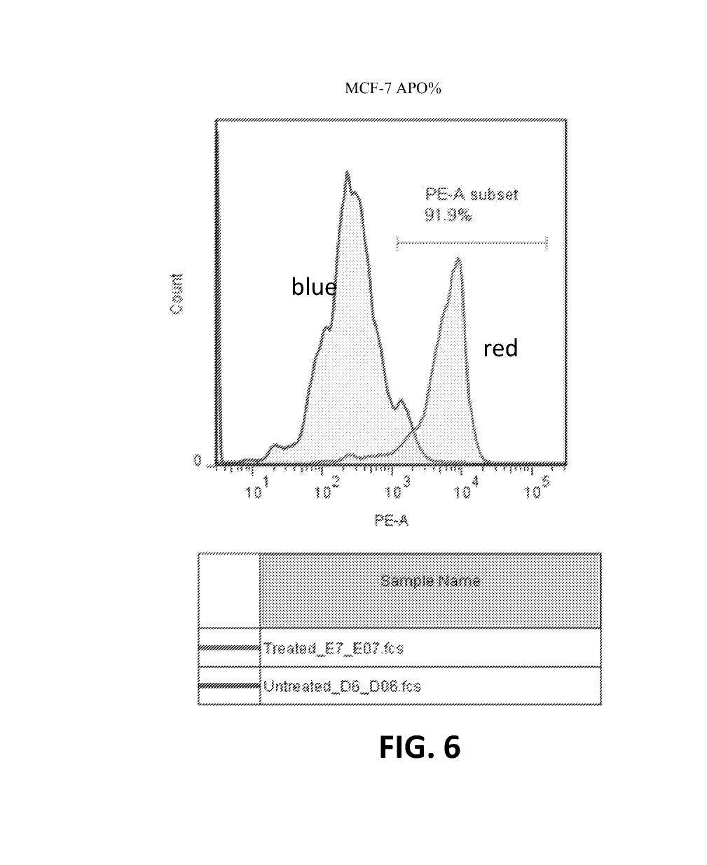

FIG. 6 and Table 2 show results of APOPercentage assay in 96-well plate on BD LSRFortessa HTS (BD Biosciences).

In FIG. 6, MCF-7 Cells untreated (blue, left) or treated (red, right) were analyzed by flow cytometry on a BD LSRFortessa HTS flow cytometer to determine the percentage of cells undergoing apoptosis. Data was analyzed using FlowJo version 7.6.5 software. Cells were seeded at a density of 5×10³ cells per well in a 96-well plate. Following day, cells were treated with 2.5 mM H₂O₂ for 30 min or 62 μl EDTA pH 6.14 after removing media for 20-30 min. as a positive control for apoptosis. Cells were lifted with EDTA and stained with 1 μl APOPercentage and analyzed by flow cytometry. 1D Histogram of untreated (blue) and treated (red) cells measuring fluorescence in the PE-A channel and applying gates to delineate Non-apoptotic (blue) and Apoptotic (red) cells.

Table 2 shows APOPercentage assay results for three cell lines tested on BD LSRFortessa HTS. Table represents data for three cell lines displaying percentage cells stained positive with APOPercentage dye in ‘Untreated’ (−ve) and ‘Treated’ (+ve) controls along with S.D.

TABLE 2

Cells stained with dye

| Cell Line | Untreated (−ve) | Treated (+ve) |
|---|---|---|
| MCF-7 | 8.96^{±1.23} | 91.9^{±1.12} |
| DU145 | 10.2^{±1.65} | 87.84^{±2.64} |
| HeLa | 11.96^{±4.02} | 91.86^{±2.79} |

Determining Mitochondrial Membrane Potential (MMP) by Flow Cytometry in Adherent Cells

A common feature of cells undergoing early stage apoptosis is disruption of mitochondria. MMP is a reflection of the permeability of the mitochondria membrane as it consists of an outer and inner membrane, which maintains a gradient of H+ ions at the inner membrane space. These H+ ions are used in the production of ATP, which serves as an energy source required for enzymatic reactions to take place and to balance energy homeostasis within the cell. Cells undergoing apoptosis often experience a decrease in ATP supply as a result of MMP disruption. Furthermore, disruption of MMP leads to the release of proapoptotic factors, such as cytochrome c, from the mitochondria into the cytosol, thus driving cells towards apoptosis. JC-1 is a membrane permeable dye with interesting fluorescence spectrum depending on the state of the dye. In healthy cells, the MMP is not disrupted and JC-1 accumulates in the mitochondria as it is attracted to the high electrochemical-gradient maintained within the inner membrane. When JC-1 accumulates it forms J aggregates, which have a fluorescence emission at 580-590 nm thus fluorescing red. In cells undergoing apoptosis, where MMP has been disrupted, JC-1 cannot aggregate due to loss of the gradient within the mitochondria and therefore remains in a monomeric state resulting in a fluorescence emission of 530 nm (green fluorescence). This unique characteristic allows distinguishing apoptotic from non-apoptotic cells at early stages of cell death when MMP is lost.

The following can be the steps used to determining MMP by Flow Cytometry in adherent cells. 5000 cells per well were plated in 45 μl of media in 96-well plate, shaken at 750 rpm with an Orbital shaker for 20 seconds, and allowed to settle 16-24 hours (h). Desired concentration of drug was added to cells in a volume of 50 to make the final volume per well up to 50 μl, was shaken at 750 rpm with an Orbital shaker for 20 seconds, and was then incubated for desired period of time. 12 μl of 15 mM EDTA pH 6.14 (upto 20 mM can be used depending on cell line) per well was added. Shake at 750 rpm with an Orbital shaker for 20 seconds and incubate at 37° C. for 15 minutes (min) to 1 h (until cells lift). Add 15 μl of serum free media containing JC-1 dye so that the final concentration is 2 μM in a total volume of 77 μl and incubate for 1-2 h at 37° C. Analyze on flow cytometer setting a threshold for FSC-H of 80 000, cytometer speed at medium (2 μl/sec), peristaltic pump speed at 23 rpm, gating on 2D plot of FSC vs SSC, performing a quadrant gate in a 2D plot of FL2-H vs FL-1H applying compensation FL-2H minus FL-1H to separate populations and a 1D plot measuring FL2-H on the X axis. 100 mM H₂O₂ can be used as a positive control.

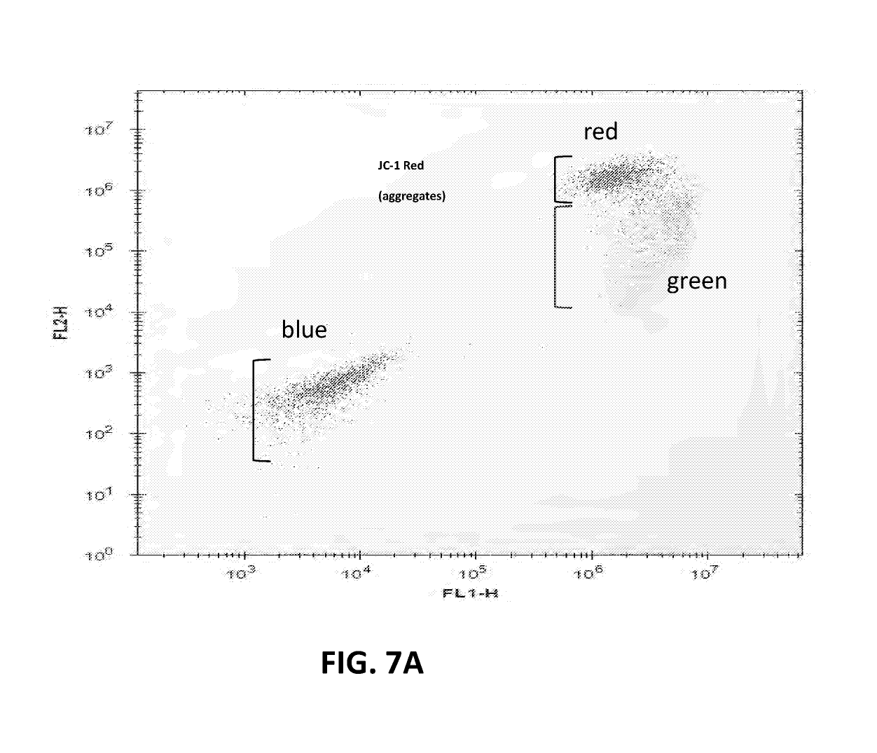

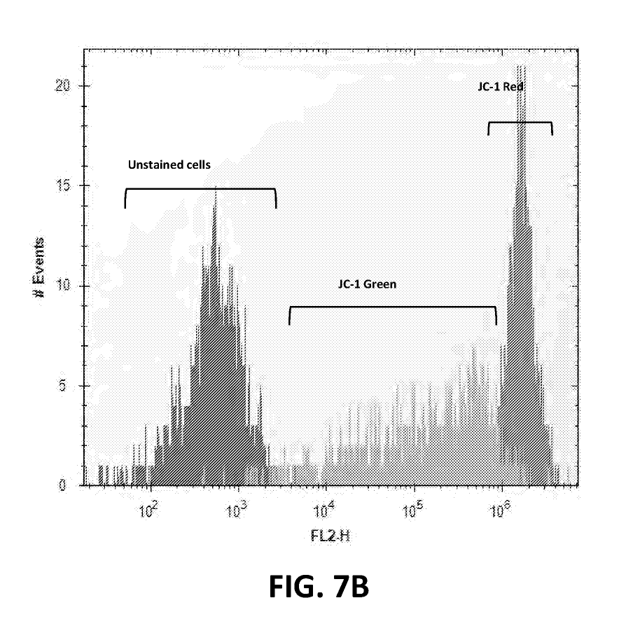

FIGS. 7A-B and Table 3 show results of MMP assay in 96-well plate on Accuri C6 HTFC System (Intellicyt).

In FIGS. 7A-B, DU145 Cells unstained (blue), untreated (red) or treated (green) were analyzed by flow cytometry on an Accuri C6 to determine the percentage of cells with disrupted MMP. Cells were seeded at a density of 5×10³ cells per well in 96-well plate. Following day media was removed from cells and replaced with 50 μl EDTA as a positive control for MMP disruption for 60 min. Cells were lifted with EDTA and stained with a final concentration of 2 μM JC-1 dye and analyzed by flow cytometry. Separation of unstained (blue), untreated (red) and treated (green) cells based on FL2-H and FL-1-H profile (A). 1D Histogram of unstained (blue), untreated (red) and treated (green) cells measuring fluorescence in the FL2-H channel (B).

Table 3 shows different aspects of method standardized using various cell lines. ‘Stability of cells in EDTA’ refers to the time limit until the percentage of cells having JC-1 green remain <10% in negative control.

TABLE 3

| | | | | Percent cells | Percent cells |
|---|---|---|---|---|---|
| | Time to lift with EDTA | | Stability in | having JC-1 green | having JC-1 green |
| | (stability of | Time to stain | JC-1 dye | in untreated | in treated |
| Cell Line | cells in EDTA) | with JC-1 | after staining | negative control | positive control |
| DU145 | 20-30 min | 60 min | 60 min+ | 10 | 90 |
| | (stable up to 2 h) | | | | |
| HeLa | 30-45 min | 60 min | 120 min | 10 | 90 |
| | (stable up to 90 mm) | | | | |
| MCF-7 | 15-20 min | 60 min | 120 min | 5 | 95 |

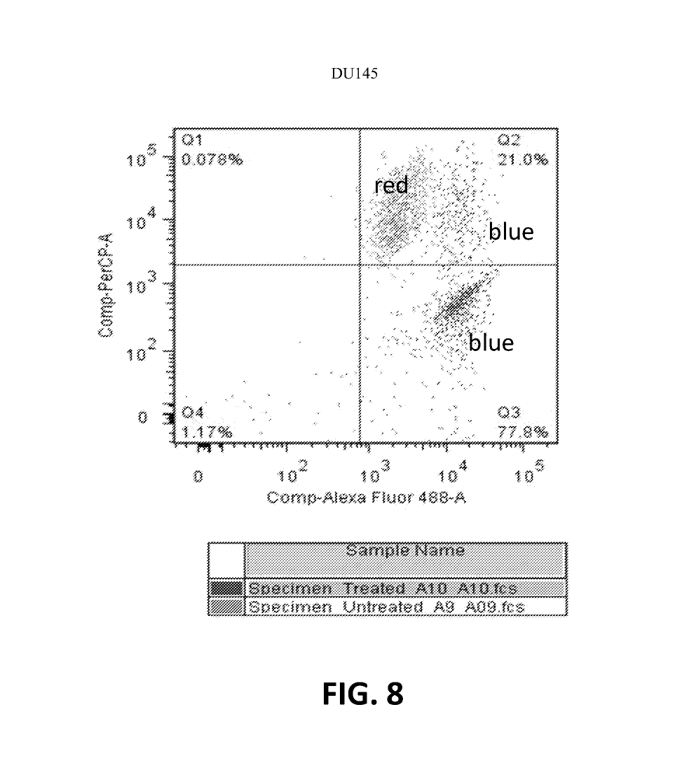

FIG. 8 and Table 4 show MMP assay in 96-well plate on BD LSRFortessa HTS (BD Biosciences).

In FIG. 8, DU145 and MCF-7 Cells were analyzed by flow cytometry on a BD LSRFortessa HTS to determine the level of MMP in cells. Data was analyzed using FlowJo version 7.6.5 software. Cells were seeded at a density of 5×10³ cells per well in a 96-well plate, the following day cells were left untreated (red) or treated with 100 mM H₂O₂ as a positive control (blue) for MMP disruption. DU145 cells were stained with a final concentration of 2 μM JC-1 dye (representative image). Cells were lifted with EDTA and analyzed by flow cytometry.

Table 4 shows MMP assay results for two cell lines tested on BD LSRFortessa HTS. Table represents median of the peaks of MMP in untreated (−ve) and treated (+ve) cells for two different cell lines.

TABLE 4

Median of cells with accumulated
JC-1 dye (Red)

| Cell Line | Untreated (−ve) | Treated (+ve) |
|---|---|---|
| MCF-7 | 4985.53^{±850.31} | 68.07^{±33.27} |
| DU145 | 12431.42^{±3885.06} | 304.23^{±37.39} |

In above experiment, JC-1 dye is accumulated in cells having intact MMP, therefore untreated negative controls show more accumulation of dye (as represented by median values in Table 4).

MMP can be measured in adherent cells in 384-well plate format. The following steps can be used. 3000 cells per well were plated in 15 μl of media in 384-well plate, centrifuged at 1000 rpm for 30 seconds, and shaken at 1500 rpm with an Orbital shaker for 20 seconds and allowed to settle 16-24 hours (h). Desired concentration of drug was added to cells in a volume of 3 μl to make the final volume per well up to 20 μl. Incubate with drug for desired period of time. Centrifuge at 1000 rpm for 30 seconds and shake at 2000 rpm with an Orbital shaker for 20 seconds. Stain cells by adding 2 μl of JC-1 to a final concentration of 2 μM in a final volume of 20 μl. Centrifuge at 1000 rpm for 30 seconds and shake at 2000 rpm with an Orbital shaker for 20 seconds and incubate with stain for 30 min at 37° C. Add 5 μl of 15 mM EDTA pH 6.14 per well (upto 20 mM can be used depending on cell line), shake at 2000 rpm with an Orbital shaker for 20 seconds and incubate at 37° C. for 55 minutes (min) to 1 h (until cells lift). Analyze on flow cytometer. The results indicate that the ratios of negative and positive controls are similar to that obtained for 96-well plate format. The assay is stable in 384-well plate thus providing platform for high throughput testing of large number of samples.

Measuring Reactive Oxygen Species (ROS) by Flow Cytometry in Adherent Cells.

Reactive oxygen species (ROS) are oxygen species with an extra electron charge e.g. O²⁻ and OH⁻. These reactive molecules are produced in cells as by-products of cellular pathways e.g. oxidative phosphorylation and play a role in cell signaling pathways. A balance of these species is required in the cell as excessive ROS can cause adverse effects and can lead to diseases like cancer, atherosclerosis and certain neurodegenerative diseases. ROS is kept in check by antioxidants like Glutathione peroxidase, catalase and superoxide dismutase (SOD), which serve to protect against ROS stress. Excessive ROS production can lead to protein unfolding, disruption of enzyme activity and also DNA damage as the available electrons in ROS can attack/reduce positively charged groups within proteins and DNA. Measuring ROS is therefore important as it gives an indication of the oxidative stress level within the cell, which might play a role in necrosis or apoptosis. 2′,7′-dichlorofluorescein diacetate (DCFDA) is a cell permeable fluorescent molecule, which is deacetylated by cellular esterases and becomes free to react with ROS, thus forming stable 2′,7′-dichlorofluorescin (DCF). DCF is stable and highly fluorescent and can be measured by flow cytometry with excitation of 495 nm and emission of 530 nm.

The following steps can be used to measure Reactive Oxygen Species (ROS) by Flow Cytometry in adherent cells. Plate 5000 cells per well in 45 μl of media in 96-well plate. Shake at 750 rpm with an Orbital shaker for 20 seconds and allow cells to settle 16-24 hours (h). Add desired concentration of drug to cells in a volume of 3 μl to make the final volume per well up to 48 μl. Add 2 μl DCFDA so that the final concentration is 10 μM in a total volume of 50 μl. Shake at 750 rpm with an Orbital shaker for 20 seconds and incubate at 37° C. and assay from 1 h onwards. Add 12 μl 15 mM EDTA pH 6.14 per well (upto 20 mM can be used depending on cell line), shake at 750 rpm with an Orbital shaker for 20 seconds and incubate at 37° C. for 15 min to 1 h (until cells lift). Add 15 μl of serum free media and incubate for 5-10 min. Analyze on flow cytometer setting a threshold for FSC-H of 80 000, cytometer speed at medium (2 μl/sec), peristaltic pump speed at 23 rpm, gating on 2D plot of FSC vs SSC and a 1D plot measuring FL1-H on the X axis. 2-10 mM H₂O₂ is used as a positive control.

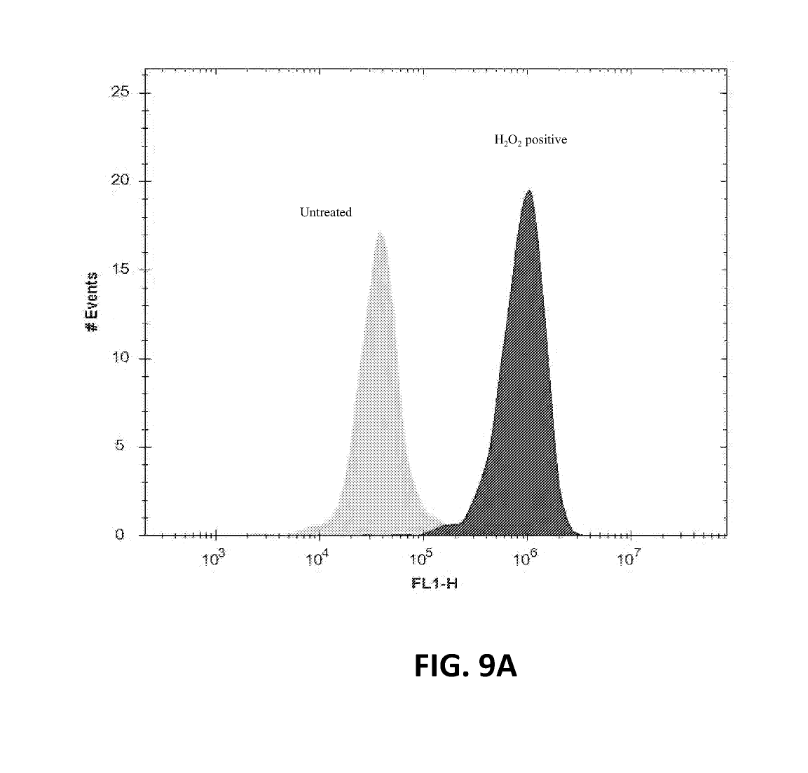

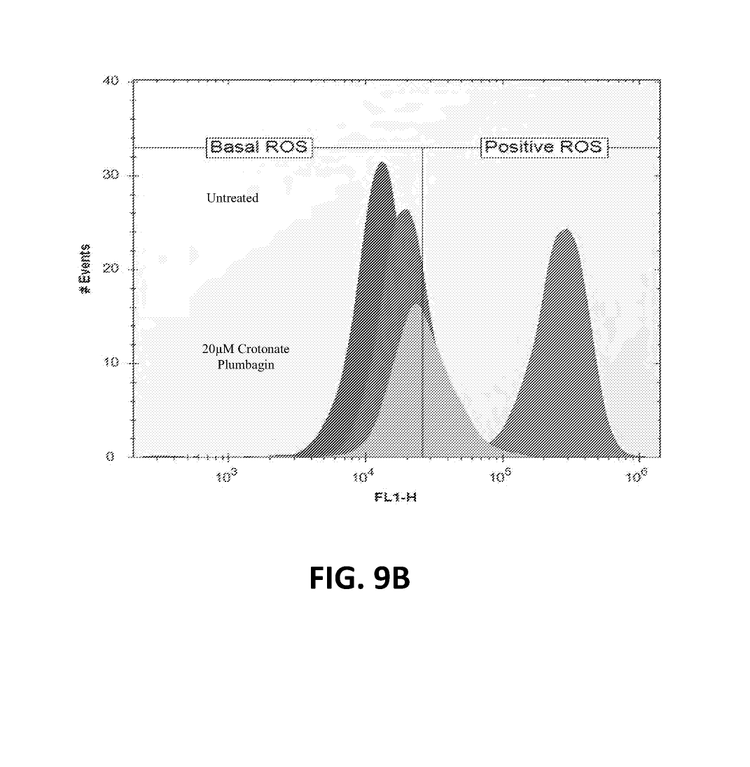

FIGS. 9A-B and Table 5 show results of ROS assay in 96-well plate on Accuri C6 HTFC System (Intellicyt).

In FIG. 9, HeLa and MCF-7 Cells were analyzed by flow cytometry on an Accuri C6 to determine the level of ROS in cells. Cells were seeded at a density of 5×10³ cells per well in a 96-well plate, the following day cells were treated with 10 mM H₂O₂ as a positive control for ROS and test compounds. HeLa cells were stained with a final concentration of 10 μM DCFDA dye immediately after adding H2O2 (A). After incubation cells were lifted with EDTA and analyzed by flow cytometry. Separation of untreated (yellow) and treated (purple) cells based on FL-1-H profile (A). MCF-7 cells were treated with Plumbagin and its derivative Crotonate Plumbagin for 1 h (B). A final 10 μM DCFDA was added simultaneously with Plumbagin and its derivative to cells. Following incubation with drug cells were lifted with EDTA and analyzed by flow cytometry.

Table 5 shows different aspects of method standardized using various cell lines. ‘Stability of cells in EDTA’ refers to the time limit until the percentage of cells stained with DCFDA remain <10% in negative control.

TABLE 5

| | | | | Percent cells | Percent cells |
|---|---|---|---|---|---|
| | Time to lift with EDTA | | | stained with DCFDA | stained with DCFDA |
| | (stability of | Time to stain | Stability in | in untreated | in treated |
| Cell Line | cells in EDTA) | with DCFDA | DCFDA dye | negative control | positive control |
| HeLa | 30-45 min | 120 min | 120 min | 10 | 90 |
| | (stable up to 90 min) | | | | |
| MCF-7 | 15-20 min | 120 min | 120 min | 10 | 90 |
| BT20 | 40-60 min | 120 min | 120 min | 5 | 95 |

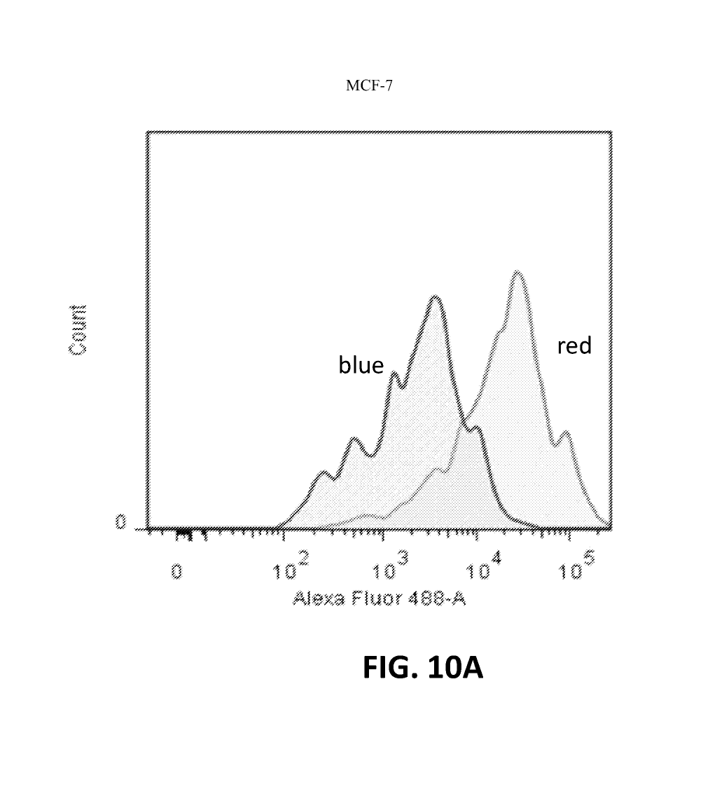

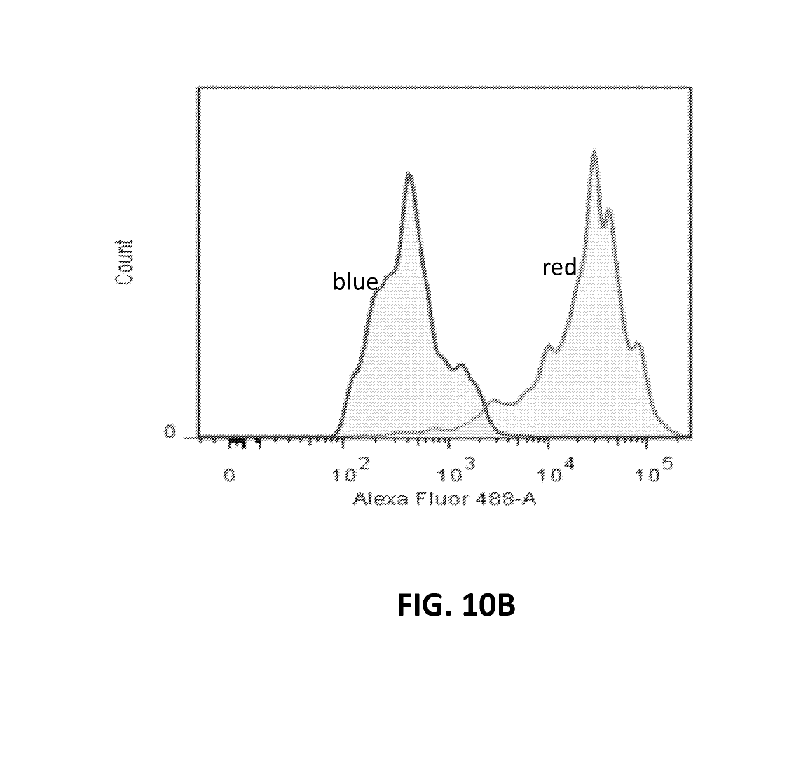

FIGS. 10A-B and Table 6 show results of ROS assay in 96-well plate on BD LSRFortessa HTS (BD Biosciences)

In FIGS. 10A-B, MCF-7 and HeLa Cells were analyzed by flow cytometry on a BD LSRFortessa HTS to determine the level of ROS in cells. Data was analyzed using FlowJo version 7.6.5 software. Cells were seeded at a density of 5×10³ cells per well in a 96-well plate, the following day cells were left untreated (blue peak) or treated with 10 mM H₂O₂ as a positive control (red peak) for ROS. MCF-7 and HeLa cells were stained with a final concentration of 10 μM DCFDA dye immediately after adding H₂O₂ (A and B). Cells were lifted with EDTA and analyzed by flow cytometry.

Table 6 shows ROS assay results for three cell lines tested on BD LSRFortessa HTS. Table 6 represents median of the peaks of ROS generation in untreated (−ve) and treated (+ve) cells for three different cell lines.

TABLE 6

ROS peak Median

| Cell Line | Untreated (−ve) | Treated (+ve) |
|---|---|---|
| MCF-7 | 2372.0^{±297.59} | 21700.0^{±3397.65} |
| DU145 | 6290.4^{±1715.77} | 34016.67^{±8076.98} |
| HeLa | 427.0^{±67.69} | 23866.67^{±4503.63} |

ROS can be measured in adherent cells in 384-well plate format. The following can be the steps. Plate 3000 cells per well in 15 μl of media in 384-well plate. Centrifuge at 1000 rpm for 30 seconds and shake at 1500 rpm with an Orbital shaker for 20 seconds and allow cells to settle 16-24 hours (h). Add desired concentration of drug to cells in a volume of 3 μl to make the final volume per well up to 20 μl. Incubate with drug for desired period of time. Centrifuge at 1000 rpm for 30 seconds and shake at 2000 rpm with an Orbital shaker for 20 seconds. Stain cells by adding 2 μl of DCFDA to a final concentration of 10 in a final volume of 20 μl. Centrifuge at 1000 rpm for 30 seconds and shake at 2000 rpm with an Orbital shaker for 20 seconds and incubate with stain for 30 min at 37° C. Add 5 μl of 15 mM EDTA pH 6.14 per well (upto 20 mM can be used depending on cell line), shake at 2000 rpm with an Orbital shaker for 20 seconds and incubate at 37° C. for 45 minutes (min) to 1 h (until cells lift). Analyze on flow cytometer.

The results indicate that the ratios of negative and positive controls are similar to that obtained for 96-well plate format. The assay is stable in 384-well plate thus providing platform for high throughput testing of large number of samples.

Measuring Autophagy by Flow Cytometry in Adherent Cells

Autophagy is a naturally occurring cellular process, which recycles proteins or amino acids, provides energy during starvation or under excessive activity, and is also recognized as another form of apoptosis. Similar to apoptosis, which involves regulators and initiators, autophagy is a controlled process. Proteins are degraded by the lysosomal pathway or recycled by endosomes. Under conditions involving stress or low levels of nutrients, autophagosomes, double membrane structures consisting of early autophagic proteins e.g. Beclin and ATG5, begin forming. These then fuse to lysosomes to form what is known as the autolysosome or autophagosome. Autophagosomes degrade proteins, damaged organelle like mitochondria and by so doing provides substrates for ATP production. When excessive autophagy (termed macrophagy/mitophagy) occurs, cells undergo apoptosis. Uncontrolled autophagy has been linked to diseases like cancer, inflammatory bowel disease and neurodegenerative disorders. Since autophagy can act as a survival pathway for cells, but its excessive activation can lead to cell death. Therefore, it is another marker to measure cell stress and apoptosis when assessing the effects of unknown drugs. Monodansylcadaverine (MDC) is a fluorescent dye with excitation 335 nm and emission 512 nm. It binds selectively to autophagosomes and can be detected by flow cytometry.

The following steps can be used. 5000 cells per well were plated in 45 μl of media in 96-well plate, shaken at 750 rpm with an Orbital shaker for 20 seconds, and allowed to settle 16-24 hours (h). Desired concentration of drug was added to cells in a volume of 50 to make the final volume per well up to 50 μl, was shaken at 750 rpm with an Orbital shaker for 20 seconds, and was then incubated for desired period of time. 12 μl of 15 mM EDTA pH 6.14 per well (upto 20 mM can be used depending on cell line) was added. Shake at 750 rpm with an Orbital shaker for 20 seconds and incubate at 37° C. for 15 minutes (min) to 1 h (until cells lift). Add 15 μl of serum free media containing MDC dye so that the final concentration is 50 μM in a total volume of 77 μl and incubate for 15 min at 37° C. Analyze on flow cytometer setting a threshold for FSC-H of 80 000, cytometer speed at medium (2 μl/sec), peristaltic pump speed at 23 rpm, gating on 2D plot of FSC vs SSC and a 1D plot measuring FL1-H on the X axis. 10 μM Z36 an inducer of autophagy is used as a positive control.

Table 7 shows the results of Autophagy assay. Table 7 shows different aspects of method standardized using various cell lines. ‘Stability of cells in EDTA’ refers to the time limit until the percentage of cells stained with MDC remain <10% in negative control.

TABLE 7

| | | | | Percent cells | Percent cells |
|---|---|---|---|---|---|
| | Time to lift with EDTA | | | stained with MDC | stained with MDC |
| | (stability of | Time to stain | Stability in | in untreated | in treated |
| Cell Line | cells in EDTA) | with MDC | MDC dye | negative control | positive control |
| HeLa | 30-45 min | 10 min | 60 min | 10 | 90 |
| | (stable up to 90 min) | | | | |
| MCF-7 | 15-20 min | 10 min | +/−30 min | 10 | 90 |
| BT20 | 40-60 min | 10 min | +/−30 min | 10 | 90 |

Comparison of Cell Number Loss Using Conventional Method and “Our Method”

The conventional method can be compared with “Our method” for estimating the cell numbers in samples undergoing flow cytometry analysis. “Our method” is an example of the method comprising adding a cell lifting solution to a media including a sample of adherent cells, and incubating the sample of adherent cells with the cell lifting solution.

Conventional method in 96-well format includes plating 3-5×10³ cells in 96-well plate, treating with drug, removing media and rinse once with trypsin, lifting cells with trypsin (5-10 minutes), adding media to neutralize trypsin, pelleting cells by centrigugation (5 minutes), removing supernatant, washing twice with plate washer, pelleting cells by centrifugation (5 minutes), resuspending cell pellets in staining solutions (15 minutes), pelleting cells by centrifugation (5 minutes), removing supernatant and resuspending cell pellets, and analyzing on flow cytometer. Total time in practice for conventional method takes more than one hour.

“Our method” includes plating 3-5×10³ cells in 96-well plate, treating with drug, adding our cell lifting solution (15-20 mM EDTA Ph 6.14) (15 minutes), adding stain (15 minutes), and analyzing on flow cytometer. Total time in practice for “our method” is 35-40 minutes. Conventional method has several wash steps, which leads to cell loss in the sample. On the contrary, “Our method” does not use any wash steps.

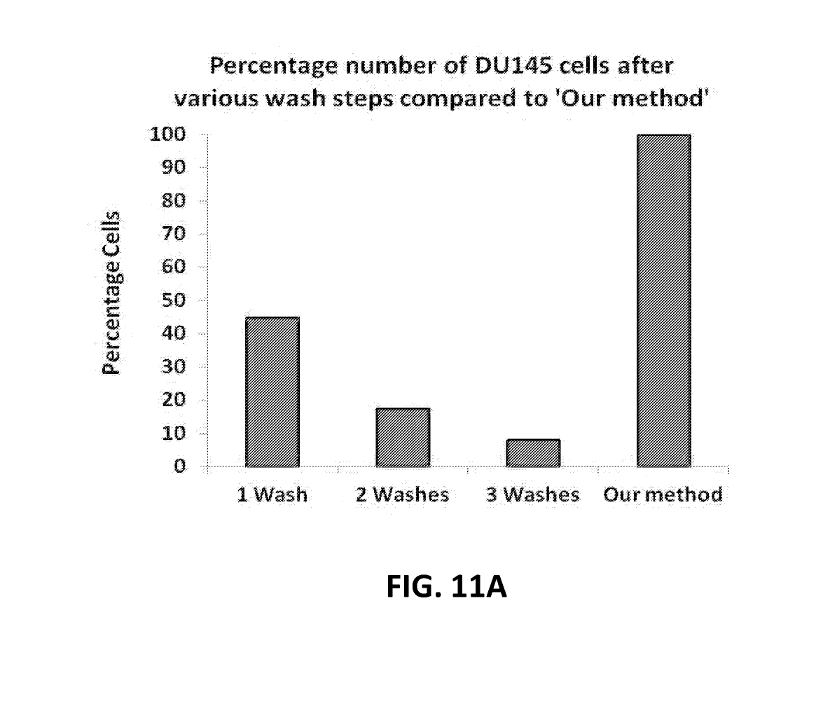

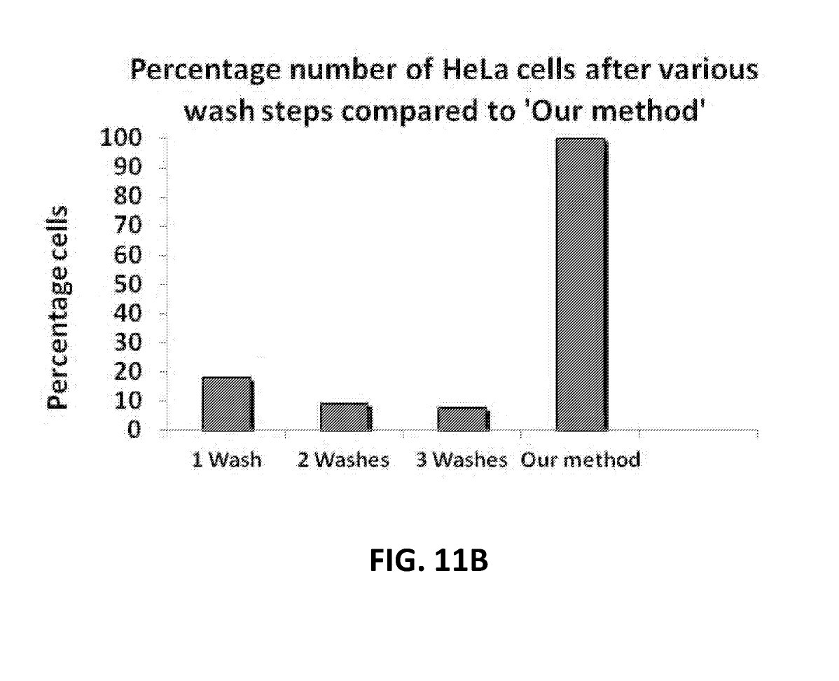

The samples using DU145 cells and HeLa cells were processed in 96-well plates using conventional method and ‘Our method’. The cell loss with each consecutive wash step using conventional protocol is shown in FIGS. 11A-B. Equal amount of final volume having suspended cells was used for flow cytometry analysis. The protocols, as shown below, were performed using two cell lines. FIGS. 11A-B shows the percentages of cells after various wash steps using conventional protocol and “Our method”. FIG. 11A shows results for DU145 cells, and FIG. 11B shows results for HeLa cells.

The described method works equally well when performed by a non-biologist user. A kit can be created with components such as buffer and APOPercentage dye, and instructions can be prepared. The method is simple and efficient that any non-skilled person in biological methods can also perform the experiments with high accuracy.

The described method has several advantages over conventional method. As shown above, conventional method can cause loss of more than 80% cells in 96-well format after two washes while ‘Our method’ retains the cells in samples as no washing is required. In “our method,” cells are grown, lifted and stained in the same well, which minimizes cell loss; there was direct staining, where cells are stained in the presence of cell lifting agent and media; no-media removal is needed, because the cell lifting solution is added directly to media; no washing of cells by using multiple centrifugation steps required; sample preparation is faster; cells are stable for longer time in the buffer, and so on. “Our method” makes High Throughput screening of adherent cells a reality.

The various embodiments described above are provided by way of illustration only and should not be construed to limit the claimed invention. Those skilled in the art will readily recognize various modifications and changes that may be made to the claimed invention without following the example embodiments and applications illustrated and described herein, and without departing from the true spirit and scope of the claimed invention, which is set forth in the following claims.

What is claimed is:

1. A method for detaching adherent cells, comprising:
adding a cell lifting solution to a media including a sample of adherent cells; and
incubating the sample of adherent cells with the cell lifting solution.

2. The method of claim 2, wherein the sample of adherent cells includes HeLa cells, DU145 cells, HT1080 cells, BT20 cells, MCF-7 cells or BJ fibroblasts.

3. The method of claim 1, wherein the sample of adherent cells is incubated with the cell lifting solution for five minutes or more.

4. The method of claim 3, wherein the sample of adherent cells is incubated with the cell lifting solution for fifteen minutes or more.

5. The method of claim 4, wherein the sample of adherent cells is incubated with the cell lifting solution for thirty minutes or more.

6. The method of claim 1, wherein the cell lifting solution includes a metal ion chelator.

7. The method of claim 6, wherein the metal ion chelator is EDTA.

8. The method of claim 1, wherein the cell lifting solution is free of enzyme.

9. A method for detaching adherent cells for flow cytometry, comprising:
adding a cell lifting solution to a media including a sample of adherent cells; and
incubating the sample of adherent cells with the cell lifting solution; and
analyzing the sample of adherent cells by flow cytometry.

10. The method of claim 9, further comprising adding an indicator of apoptosis to the sample of adherent cells.

11. The method of claim 10, wherein the indicator of apoptosis, ROS, MMP and autophagy is a dye, a fluorophore or an antibody.

12. The method of claim 10, further comprising adding a stimulus to the media.

13. The method of claim 12, wherein the stimulus is a drug.

14. The method of claim 9, wherein the adherent cells are not washed with a washing solution between adding the cell lifting solution to the media and analyzing the adherent cells by flow cytometry.

15. The method of claim 9, wherein the time period between adding the cell lifting solution to the media and analyzing the adherent cells by flow cytometry is four hours or less.

16. The method of claim 9, wherein the time period between adding the cell lifting solution to the media and analyzing the adherent cells by flow cytometry is three hours or less.

17. The method of claim 9, wherein the time period between adding the cell lifting solution to the media and analyzing the adherent cells by flow cytometry is two hours or less.

18. The method of claim 9, wherein the time period between adding the cell lifting solution to the media and analyzing the adherent cells by flow cytometry is one hour or less.

19. The method of claim 9, wherein the time period between adding the cell lifting solution to the media and analyzing the adherent cells by flow cytometry is 15-30 minutes or less.

20. The method of claim 12, wherein the stimulus is added to the media prior to adding the cell lifting solution to the media.

21. The method of claim 9, wherein the cell lifting solution includes a metal ion chelator.

22. The method of claim 9, wherein the metal ion chelator is EDTA.

23. A method of collecting adherent cells, comprising:
plating a first sample of adherent cells in media on a cell plate;
plating a second sample of adherent cells in media on a cell plate, wherein the second sample of adherent cells includes a second number of adherent cells;
adding a control buffer to the media of the first sample of adherent cells;
adding a stimulus to the media of the second sample of adherent cells;
adding cell lifting solution to the media of the first sample of adherent cells and to the media of the second sample of adherent cells, wherein the first sample includes a first number of adherent cells at the time the cell lifting solution is added to the media and the second sample includes a second number of adherent cells at the time the cell lifting solution is added to the media;
incubating the first and second samples of adherent cells, thereby detaching the first and second samples of adherent cells from the cell plate;
collecting the first sample of adherent cells, wherein the number of adherent cells collected from the first sample is equal to 75% or more of the number of adherent cells in the first sample when the cell lifting solution was added; and
collecting the second sample of adherent cells, wherein the number of adherent cells collected from the second sample is equal to 75% or more of the number of adherent cells in the second sample when the cell lifting solution was added.
